(19) United States
(12) Patent Application Publication
(10) Pub. No.: US 2007/0025938 A1
(43) Pub. Date: Feb. 1, 2007

(54) Depigmentation method and kit

(75) Inventors: Isabelle Hansenne, Westfield, NJ (US); Hani Fares, Somerset, NJ (US); Marc Cornell, Jackson, NJ (US); Sidney Peter Foltis, Nutley, NJ (US)

Correspondence Address: LERNER, DAVID, LITTENBERG,;KRUMHOLZ & MENTLIK, 600 SOUTH AVENUE WEST, WESTFIELD, NJ, 07090, US

(73) Assignee: L'OREAL, Paris (FR)

(21) Appl. No.: 11/192,481

(22) Filed: Jul. 29, 2005

(51) Int. Cl. A61K 8/365; A61K 8/31

(52) U.S. Cl. 424/62; 424/401

(57) ABSTRACT

The present invention relates to a cosmetic kit for depigmenting human skin comprising a multi-unit receptacle containing a first unit containing a microdermabrasion composition; a second unit containing a skin depigmenting composition; a third unit containing ascorbic acid in powder form; a fourth unit containing a skin soothing composition; optionally, a fifth unit containing a maintenance composition, and optionally a sixth unit containing a photoprotective composition. The present invention also relates to a method for treating human skin in need of depigmentation comprising the successive application, to an area of skin, of the compositions contained in the multi-unit receptacle.

### BACKGROUND OF THE INVENTION

In humans, skin color develops as the result of a series of complex cellular processes that are carried out within cells called melanocytes. Melanocytes are located in the lower layer of the skin's epidermis and their function is to produce melanin, a pigment which protects the body from the damages caused by the ultraviolet (UV) rays from the sun.

Melanogenesis, the process by which melanin is produced, involves several steps, from tyrosine to L-DOPA (Dihydroxyphenylalanine) to Dopaquinone to Dopachrome to Melanins. The first two steps of this process are catalyzed by tyrosinase. The activity of this enzyme is under the influence of the α-melanocyte stimulating hormone and UV rays.

Typically, the production of melanin by melanogenesis leads to a darker skin tone such as a tan. However, undesirable pigmentation may occur, such as the development of age spots, liver spots, lentigines or hyperpigmentation.

The people with these undesirable pigmentation patterns or those who desire a whiter skin tone therefore require the use of depigmenting compositions. While the process can be done at a dermatologist's office, there is a need for self application at home.

### BRIEF SUMMARY OF THE INVENTION

The present invention relates to a cosmetic kit for depigmenting human skin comprising a multi-unit receptacle containing:
(a) at least one unit containing a microdermabrasion composition having at least one type of skin abrading particle;
(b) at least one unit containing a skin depigmenting composition having at least one whitening agent and, optionally, at least one skin peeling agent;
(c) optionally, at least one unit containing at least one skin peeling agent;
(d) at least one unit containing a skin soothing composition having at least one skin soothing agent;
(e) optionally, at least one unit containing a maintenance composition having at least one skin whitening agent; and
(f) optionally, at least one unit containing a photoprotective composition.

Yet another embodiment of the present invention relates to a cosmetic kit for depigmenting human skin comprising a multi-unit receptacle containing:
(a) at least one unit containing a microdermabrasion composition having at least one type of skin abrading particle;
(b) at least one unit containing a depigmenting composition having at least one skin whitening agent, at least one skin peeling agent and at least one skin soothing agent;
(c) optionally, at least one unit containing a maintenance composition having at least one skin whitening agent and, optionally, at least one skin peeling agent; and
(d) optionally, at least one unit containing a photoprotective composition.

According to another embodiment of the present invention, there is provided a method for depigmenting human skin involving the steps of:
(a) providing a unit containing a microdermabrasion composition having at least one type of skin abrading particle;
(b) providing a unit containing a skin depigmenting composition having at least one skin whitening agent and, optionally, at least one skin peeling agent;
(c) optionally, providing a unit containing at least one skin peeling agent;
(d) providing a unit containing a skin soothing composition having at least one skin soothing agent;
(e) optionally, providing a unit containing a photoprotective composition;
(f) contacting the skin with the microdermabrasion composition to form abraded skin;
(g) removing the microdermabrasion composition from the abraded skin;
(h) contacting the skin with the skin depigmenting composition and, optionally, the skin peeling agent;
(i) applying a layer of skin soothing composition over the skin depigmenting composition;
(j) allowing the skin- depigmenting composition and the skin soothing composition to remain on the skin for a predetermined period of time;
(k) removing the skin depigmenting composition and the skin soothing composition from the skin; and
(l) applying the photoprotective composition onto the skin.

This method is used for an amount of time sufficient for achieving depigmenting of the targeted area of skin.

### DETAILED DESCRIPTION OF THE INVENTION.

Other than in the operating examples, or where otherwise indicated, all numbers expressing quantities of ingredients are to be understood as being modified by the term “about”.

The Microdermabrasion Composition

The microdermabrasion composition contains at least one type of skin abrading particle.

Suitable skin abrading particles for use in the present invention include, but are not limited to, metal oxide particles, pumice, polyethylene, jojoba, diatomaceous earth, silica, sugar, ground fruit kernels and wax beads.

Metal oxide particles may include magnesium oxide or aluminum oxide. These particles have a purity of at least 95%, better still of at least 99%. Their average particle size preferably ranges from 100 to 180 μm. Aluminum oxide is preferred for use in the present invention, in particular in the anhydrous crystalline form (corundum).

Aluminum oxide particles are available as particles calcined at high temperature, until the α-Al₂O₃ crystalline structure of corundum is obtained, and then treated in order to form grains having sharp edges and having a given particle size distribution, the particles preferably having an average particle diameter of between 100 and 180 μm and preferably between 130 and 150 μm. Their distribution is preferably such that none of the particles has a diameter greater than 250 μm. Such particles are available commercially in particular from MarkeTech International under the trade name Dermagrain™. The particles referenced as Dermagrain™ 900 are composed of crystalline α-alumina with a purity of 99.55% having an average particle size diameter of approximately 140 μm, the particles all having a diameter of less than 250 μm. Less than 3% of the particles have a diameter of less than 105 μm. Other particles are available from Industrial Supply under the trade names ARL 100 and ARL 120. They are aluminum oxide particles having an average particle diameter of 120 and 100 μm respectively and a particle size distribution ranging from 75 to 212 μm and from 63 to 180 μm respectively.

In another embodiment, the metal oxide particles used in the composition according to the invention may be magnesium oxide preferably having an average particle size ranging from 100 to 180 μm. An example of particles of this type is sold by MarkeTech International under the trade name Magnaderm™ 100. They are particles having an average particle diameter of approximately 120 μm and a purity of at least 99%.

In another embodiment the particles used in the microdermabrasion composition may be ground up fruit kernel powder, such as apricot, peach or nectarine kernel powder, or diatomaceous earth. These powders are available commercially from companies such as Alban Muller.

The skin abrading particles will be present in an amount sufficient to abrade the targeted area of skin. Preferably, the skin abrading particles may be present in concentrations ranging from 0.5 to 40%, preferably from 10 to 30% and more preferably from 15 to 25% by weight, based on the total weight of the microdermabrasion composition. This composition preferably has a pH of greater than 4 and less than 8, better still less or equal to 7 and even better still of between 5.5 and 7.

The microdermabrasion composition contained in the first unit of the multi-unit receptacle according to the invention may be provided in the form of a lotion, gel, fluid or cream. It may comprise various adjuvants and advantageously may include at least one heterogeneous polysaccharide. This is because the Applicant has demonstrated that these compounds make it possible to reduce the discomfort associated with the use of the abovementioned abrasive metal oxide particles. This heterogeneous polysaccharide can be an alginate or alternatively a heterogeneous polysaccharide comprising at least one fucose unit, comprising in particular fucose, galactose and galacturonic acid units, in particular a linear sequence of α-L-fucose, of α-D-galactose and of galacturonic acid. Such a polysaccharide is available in particular in the form of a 1% solution in water from Solabia under the trade name Fucogel 1000 PP®.

This composition may be applied to the skin by manual massaging with the fingertips or by mechanical massaging using a vibrating device provided with a massaging head equipped with a pad, as disclosed in Application US2001/0046506 or U.S. Patent No. 6,652,888, for example.

The Skin Depigmenting Composition

The skin depigmenting composition contains at least one skin whitening agent, present in an amount sufficient to whiten the skin.

Suitable skin whitening agents include, but are not limited to: phenolic derivatives, such as hydroquinone, hydroquinone monoethyl ether, hydroquinone monobenzyl ether or 4-hydroxyanisole; kojic acid and its derivatives; azelaic acid and its derivatives; linoleic acid; resorcinol and its derivatives such as those described in WO 00/56702, the entire contents of which are incorporated herein by reference; ellagic acid; zinc peroxide; mercury chloride; arbutin and its derivatives such as those described in applications EP 895 779 and EP 524 109, the entire contents of which are incorporated herein by reference; extracts of Emblica officinalis such as those described in U.S. Pat. No. 6,261,605, and in pages 175 and 176 of the 1990 Handbook of Ayurvedic Medicinal Plants by L.D. Kapoor, CRC Press, Inc., the entire contents of which are herein incorporated by reference; aminophenol derivatives such as those described in applications WO 99/10318 and WO 99/32077, the entire contents of which are incorporated herein by reference, and in particular N-cholesteryloxy-carbonyl-para-aminophenol and N-ethyloxycarbonyl-para-aminophenol; dihydroxybenzene derivatives such as those described in WO 00/47045, the entire contents of which are incorporated herein by reference; guaiacol derivatives such as those described in WO 00/47179, the entire contents of which are incorporated herein by reference; 4-(2,3-dihydroxyphenyl)cyclohexanol such as described in WO 00/56279, the entire contents of which are incorporated herein by reference; iminophenol derivatives, in particular those described in application WO 99/22707, the entire contents of which are incorporated herein by reference; L-2-oxothiazolidine-4-carboxylic acid or procysteine, and its salts and esters; and extracts of plants, in particular of liquorice, mulberry and skullcap. Hydroquinone, ascorbic acid and their mixtures are preferred for use in the present invention.

The skin whitening agent may preferably be present in an amount of from 0.1 to 10% by weight, preferably 1 to 5% by weight, and more preferably 1.5 to 3% by weight, based on the total weight of the skin depigmenting composition.

The Skin Peeling Agent

A skin peeling agent may also be used in order to boost the efficiency of the skin whitening agent. Without intending to be bound by theory, it is believed that skin peeling agent stimulates collagen production in the skin, thereby further enhancing the skin depigmentation phenomenon.

Suitable skin peeling agents include, but are not limited to: hydroxy acids and their derivatives, such as ascorbic, citric, lactic, glycolic, malic, tartaric, mandelic, salicylic and its derivatives such as 5-(n-octanoyl)salicylic acid (also known as Capryloyl Salicylic Acid under the CTFA designation), examples of which are described in U.S. Pat. No. 4,767,750, the entire contents of which is hereby incorporated by reference; urea; aminosulphonic compounds and in particular N-(2-hydroxyethyl)piperazine-N′-2-ethanesulphonic acid (HEPES); and derivatives of 2-oxothiazolidine-4-carboxylic acid (procysteine). Particularly preferred skin peeling agents for use in the present invention include glycolic, ascorbic and capryloyl salicylic acid.

The skin peeling agent may be present in its own unit, in which case it will be used in an amount of from 1 to 100% by weight, preferably 5 to 50% by weight, and more preferably 5 to 15% by weight, based on the total weight of its own unit. On the other hand, the skin peeling agent may also be present in the skin depigmenting composition, in which case it will be used in an amount of from 0.5 to 70% by weight, preferably 5 to 50% by weight, and more preferably 5 to 15% by weight, based on the total weight of the skin depigmenting composition.

According to one embodiment of the invention, the skin depigmenting composition contains 2% hydroquinone as the skin whitening agent and 10% glycolic acid as the skin peeling agent.

According to another embodiment of the invention, an individual unit containing a skin peeling agent is provided, preferably powder-form ascorbic acid, in an amount of at least 90% by weight, based on the weight of the individual unit.

The Skin Soothing Composition

The skin soothing composition contains at least one skin soothing agent, present in an amount sufficient to both soothe previously weakened skin and protect it from further assaults, particularly those from the environment.

The skin soothing composition according to the invention will contain at least one skin soothing agent conventionally used to soothe skin. Examples thereof include, but are not limited to, emollients such as silicones, and hydrating agents such as polyols, i.e. glycerin and/or propylene glycol which may be homogenized together in the presence of an emulsifier. It may also comprise additives such as at least one antioxidant for combating free radicals, such as tocopherol, tocopherol acetate, ascorbic acid and arginine pyrrolidonecarboxylate.

The skin soothing agent will typically be present in the composition in an amount of from 1 to 40% by weight, preferably from 3 to 35% by weight, and more preferably from 5 to 25% by weight, based on the weight of the composition. A particularly preferred skin soothing agent is silicone.

The skin soothing composition is generally used in the form of a cream and may be advantageously dispensed from a flexible tube, a pot or any other suitable type of container.

The Maintenance Composition

For some people, the microdermabrasion and depigmenting steps may result in excessive skin irritation. It may then be recommended to skip these steps every other day and replace them with the application of a maintenance composition. The maintenance composition preferably contains both a skin peeling agent and, optionally, a skin whitening agent in concentrations such that the maintenance composition, while being effective as a depigmentation composition, will be milder on the skin. In a particularly preferred embodiment, the maintenance composition contains at least one skin whitening agent in an amount less than that of the skin depigmenting composition and, optionally, at least one skin peeling agent. In a preferred embodiment, the maintenance composition contains 2% by weight of hydroquinone and 0.4% by weight of capryloyl salicylic acid, all weights based on the weight of the maintenance composition. In another preferred embodiment of the invention, the maintenance composition may further contain sunscreens. In a particularly preferred embodiment, the maintenance composition contains 2% by weight of hydroquinone, 0.4% by weight of capryloyl salicylic acid and sunscreens in a concentration sufficient to provide an SPF value of at least 15, all weights based on the weight of the maintenance composition.

The Photoprotective Composition

It is highly recommended that a photoprotective composition, preferably one having an SPF of at least 15, be applied onto treated skin in order to protect it from the harmful effects of the sun.

Suitable organic photoprotective agents may be chosen from anthranilates; cinnamic derivatives; dibenzoylmethane derivatives; salicylic derivatives; camphor derivatives; triazine derivatives, such as those disclosed in patents and patent applications U.S. Pat. No. 4,367,390, EP 863 145, EP 517 104, EP 570 838, EP 796 851, EP 775 698, EP 878 469, EP 933 376, EP 507 691, EP 507 692, EP 790 243 or EP 944 624, the entire contents of which are incorporated herein by reference; benzophenone derivatives; β,β-diphenylacrylate derivatives; benzotriazole derivatives; benzalmalonate derivatives; benzimidazole derivatives; imidazolines; bisbenzoazolyl derivatives, such as disclosed in Patents EP 669 323 and U.S. Pat. No. 2,463,264, the entire contents of which are incorporated herein by reference; p-aminobenzoic acid (PABA) derivatives; methylenebis(hydroxyphenylbenzotriazole) derivatives, such as disclosed in patents and patent applications U.S. Pat. Nos. 5,237,071, 5,166,355, GB 2,303,549, DE 19 726 184 and EP 893 119, the entire contents of which are incorporated herein by reference; benzoxazole derivatives, such as disclosed in Patent Applications EP 832 642, EP 1 027 883, EP 1 300 137 and DE 10 162 844; screening polymers and screening silicones, such as those disclosed in particular in patent application WO 93/04665, the entire contents of which are incorporated herein by reference; dimers derived from α-alkylstyrene, such as those disclosed in patent application DE 19 855 649, the entire contents of which are incorporated herein by reference; 4,4-diarylbutadienes, such as disclosed in Applications EP 967 200, DE 19 746 654, DE 19 755 649, EP 1 008 586, EP 1 133 980 and EP 1 133 981, the entire contents of which are incorporated herein by reference, and their mixtures.

The organic photoprotective agents more particularly preferred may be chosen from the following compounds (CTFA names or chemical names):
Ethylhexyl Salicylate,
Ethylhexyl Methoxycinnamate,
Octocrylene,
Phenylbenzimidazole Sulfonic Acid,
Benzophenone-3,
Benzophenone-4,
Benzophenone-5,
4-Methylbenzylidene Camphor,
Terephthalylidene Dicamphor Sulfonic Acid,
Disodium Phenyl Dibenzimidazole Tetrasulfonate,
2,4,6-Tris(diisobutyl 4′-aminobenzalmalonate)-s-triazine,
Anisotriazine,
Ethylhexyl Triazone,
Diethylhexyl Butamido Triazone,
Methylene Bis-Benzotriazolyl Tetramethyl-butylphenol,
Drometrizole Trisiloxane,
Polysilicone-15,
1,1-Dicarboxy (2,2′-dimethylpropyl)-4,4-diphenylbutadiene,
2,4-Bis[5-1(dimethylpropyl)benzoxazol-2-yl-(4-phenyl)imino]-6-(2-ethylhexyl)imino-1,3,5-triazine, and their mixtures.

Suitable inorganic photoprotective agents may be chosen from pigments or alternatively nanopigments (mean size of the primary particles: generally between 5 nm and 100 nm, preferably between 10 nm and 50 nm) formed of metal oxides which may or may not be coated, such as, for example, nanopigments formed of titanium oxide (amorphous or crystalline in the rutile and/or anatase form), iron oxide, zinc oxide, zirconium oxide or cerium oxide, which are all UV photoprotective agents well known per se. Furthermore, conventional coating agents are alumina and/or aluminum stearate. Such nanopigments formed of metal oxides, which may or may not be coated, are disclosed in particular in Patent Applications EP 518 772 and EP 518 773, the entire contents of which are incorporated herein by reference.

The photoprotective agents can be present in the photoprotective composition in amounts ranging from 2 to 35% by weight, and preferably from 5 to 30% by weight, based on the weight of the photoprotective composition. The photoprotective composition preferably has an SPF value of at least 15, such as 20 or 30 or greater.

In order to enhance soothing and/or to lessen irritation, the skin soothing composition and/or the maintenance composition may be formulated with thermal or mineral waters. By “thermal or mineral water” is meant water having a mineral content of at least 300 mg/l.

In this respect, the term “mineral content” is understood to mean the sum of the concentrations of anions and of cations present in the thermal or mineral water. The fact of using a water with a high mineral content makes it possible to compensate for the irritating effect of the microdermabrasion and the skin depigmenting compositions employed previously.

In the present invention, use is made without distinction of a thermal water or of a mineral water. Generally, a mineral water is suitable for consumption, which is not always the case with a thermal water. Each of these waters comprises, inter alia, trace elements and dissolved minerals.

The thermal and/or mineral water used according to the invention can have a mineral content of at least 400 mg/l, in particular of at least 700 mg/l, and more particularly a total concentration of carbonates and of bicarbonates of at least 150 mg/l and more preferably of at least 360 mg/l and in particular of sodium carbonate and bicarbonate of greater than 2 mg/l. The concentration of silicon oxide in the water used in the composition according to the invention can preferably be at least 6 mg/l and more preferably at least 9 mg/l.

The thermal water or the mineral water used according to the invention can be chosen from water from Avène, water from Vittel, waters from the Vichy basin, water from Uriage, water from La Roche Posay, water from La Bourboule, water from Enghien-les-Bains, water from Saint Gervais-les-Bains, water from Néris-les-Bains, water from Allevard-les-Bains, water from Digne, water from Maizières, water from Neyrac-les-Bains, water from Lons-le-Saunier, water from Eaux-Bonnes, water from Rochefort, water from Saint Christau, water from Les Fumades and water from Tercis-les-Bains.

Among these waters, those which exhibit a mineral content of less than 700 mg/l but of greater than 400 mg/l are water from La Roche Posay, water from Eaux-Bonnes or water from Saint Christau.

Among these waters, those which exhibit a total concentration of carbonates or bicarbonates of greater than 360 mg/l are water from Vittel, water from La Bourboule, water from Les Fumades, water from Enghien-les-Bains, water from La Roche Posay, water from the Vichy basin or water from Uriage.

Among these waters, those which exhibit a concentration of carbonates or bicarbonates of between 150 mg/l and 360 mg/l are water from Digne, water from Maizières, water from Rochefort or water from Saint Gervais-les-Bains.

Among these waters, those which comprise at least 2 mg/l of sodium carbonate or bicarbonate are water from La Roche Posay, water from Vittel, waters from the Vichy basin and water from Uriage.

The waters comprising at least 9 mg/l of silicon oxide are water from La Roche Posay, water from Vittel, waters from the Vichy basin or water from Uriage.

Waters from the Vichy basin are preferred for use in the present invention.

The water used in the skin soothing composition and/or the maintenance composition may be water suitable for use in cosmetic or dermatological compositions, which may be exclusively or partially a mineral or thermal water as defined above. The skin soothing composition and/or maintenance composition according to the invention generally may thus comprise no mineral or thermal water, more than 2% by weight, preferably more than 5% by weight, more preferably more than 7% by weight, indeed even make up all the water contained in the skin soothing composition and/or maintenance composition.

The compositions may be provided in all dosage forms conventionally used for topical application and, in particular, in the form of: (i) dispersions of the lotion or gel type, (ii) emulsions with a liquid or semi-liquid consistency of the milk type, obtained by dispersion of a fatty phase in an aqueous phase (O/W) or vice versa (W/O), (iii) suspensions or emulsions with a soft, semi-solid or solid consistency of the cream or gel type, (iv) multiple emulsions (W/O/W or O/W/O), (v) microemulsions, (vi) vesicular dispersions of ionic and/or nonionic type, or (vii) wax/aqueous phase dispersions. These compositions are prepared according to methods known to those of ordinary skill in the art of cosmetics or dermatological formulations.

The oils present in these emulsions can be silicone oils, which may be volatile or nonvolatile, hydrocarbon oils or vegetable oils. These emulsions can additionally comprise non-oily fatty substances, such as shea butter, silicone gums, esters of fatty acids and of fatty alcohols, fatty acids and fatty alcohols.

These compositions can additionally comprise various adjuvants commonly used in the cosmetics field, such as emulsifiers, including glyceryl fatty acid esters, sugar fatty acid esters, sorbitan fatty acid esters, polyethylene glycol fatty acid esters, ethoxylated fatty alcohols and alkylpolyglycosides; fillers, in particular polyacrylamide (Nylon) fibers and/or microbeads, silica, optionally in the form of a colloidal dispersion, and/or organic microspheres which are optionally expanded; preservatives and/or copreservatives, such as caprylyl glycol; sequestering agents, such as EDTA salts; colorants; fragrances; pH adjusters, such as neutralizing agents and/or buffering agents; ethanol; and thickening and gelling agents, in particular acrylamide homo- and co-polymers, acrylic homo- and co-polymers, acrylamidomethylpropanesulphonic acid (AMPS) homo- and co-polymers, and xanthan gum.

Of course, a person skilled in the art will take care to choose this or these possible additional compounds and/or their amounts so that the advantageous properties of the compositions according to the invention are not, or not substantially, detrimentally affected by the envisaged addition.

A particularly preferred embodiment of the present invention is a modified cosmetic kit containing:
(a) a unit containing a microdermabrasion composition with 20% by weight, based on the weight of the microdermabrasion composition, of aluminum oxide particles;
(b) a unit containing a skin depigmenting composition with 2% by weight of hydroquinone and 10% by weight of glycolic acid, all weights based on the weight of the first skin depigmenting composition;
(c) a dropper for dispensing the skin depigmenting composition;
(d) a unit containing a skin peeling composition, in powder form, with at least 90% by weight, based on the weight of the skin peeling composition, of ascorbic acid;
(e) a scoop for dispensing the skin peeling composition;
(f) a mixing cup for blending the skin depigmenting composition and the skin peeling composition to form a blended composition;
(g) a brush for applying the blended composition onto a targeted area of skin;
(h) a unit containing a skin soothing composition; and
(i) a unit containing a maintenance composition.

The present invention also provides for a method of treating skin in need of depigmentation using any of the above-described cosmetic kits. The method involves the steps of:
(a) providing a unit containing a microdermabrasion composition having at least one type of skin abrading particle;
(b) providing a unit containing a skin depigmenting composition having at least one skin whitening agent and, optionally, at least one skin peeling agent;
(c) providing a unit containing a skin soothing composition having at one skin soothing agent;
(d) optionally, providing a unit containing at least one skin peeling agent;
(e) optionally, providing a unit containing a maintenance composition having at least one skin whitening agent;
(f) optionally, providing a unit containing a photoprotective composition;
(g) contacting the skin with the microdermabrasion composition to form abraded skin;
(h) removing the microdermabrasion composition from the abraded skin;
(i) contacting the skin with the skin depigmenting composition and, optionally, the skin peeling agent;
(j) applying a layer of skin soothing composition over the skin depigmenting composition;
(k) allowing the skin depigmenting composition and the skin soothing composition to remain on the skin for a predetermined period of time;
(l) removing the skin depigmenting composition and the skin soothing composition from the skin; and
(m) applying the photoprotective composition onto the skin.

The precise period of time which the skin depigmenting composition and the skin soothing composition remain on the skin will depend upon a number of variables including, but not limited to, the concentration of actives contained in the compositions, as well as the desired effect being sought. However, in all instances, said period of time will be apparent to those skilled in the art.

The present invention also provides for a method of treating skin involving the steps of:
(a) providing a unit containing a microdermabrasion composition having at least one type of skin abrading particle;
(b) providing a unit containing a skin depigmenting composition having at least one skin whitening agent, at least one skin peeling agent and at least one skin soothing agent;
(c) optionally, providing a unit containing a photoprotective composition;
(d) optionally, providing a unit containing a maintenance composition having at least one skin whitening agent;
(e) contacting the skin with the microdermabrasion composition to form abraded skin;
(f) removing the microdermabrasion composition from the abraded skin;;
(g) contacting the skin with the skin depigmenting composition;
(h) allowing the skin depigmenting composition to remain on the skin for a predetermined period of time;
(i) removing the skin depigmenting composition from the skin; and
(j) applying a photoprotective composition onto the skin.

The cosmetic kits and methods of the present invention may be used on either a daily basis or on alternate days depending on the concentration of ingredients contained in the kit and/or the desired skin treating application. A particularly preferred period of use is 28 days. Variations thereof, however, will be apparent to those of ordinary skill of the art.

In order to minimize the irritation potential associated with the use of the cosmetic kit of the present invention, it is preferred to use the maintenance composition by itself, on alternate days, in place of the other components of the cosmetic kit. Thus, in use, a person may apply all of the components of a cosmetic kit with the exception of the maintenance composition on days 1, 3, 5, . . . , and use the maintenance composition by itself on days 2, 4, 6, . . . , over a period of 28 days. Daily use of the photoprotective composition is strongly recommended.

The present invention will be better understood from the examples which follow, all of which are intended for illustrative purposes only and are not meant to unduly limit the scope of the invention in any way.

The invention will now be illustrated by the following nonlimiting examples. In these examples, the amounts are shown as percentage by weight.

Microdermabrasion Composition

A microdermabrasion composition in accordance with the present invention was prepared having the following formulation:

| Ingredients (CTFA Designation) | % w/w |
|---|---|
| Ammonium Polyacryloyldimethyl Taurate | 0.8000 |
| Glycerin | 5.0000 |
| Preservatives | 1.1000 |
| Butyrospermum Parkii (Shea Butter) | 2.0000 |
| Dimethicone | 2.0000 |
| Glyceryl Stearate/PEG-100 Stearate | 3.0000 |
| Stearyl Alcohol | 1.5000 |
| Mineral Oil | 10.0000 |
| Aluminum oxide | 20.0000 |
| Dye | 0.0005 |
| Water | q.s. 100.000 |

The microdermabrasion composition was applied in the evening to a wetted face, avoiding the outline of the eyes and lips, by massaging with the fingertips for approximately 1 to 2 minutes, followed by rinsing with water and by drying using a clean towel. The application of the microdermabrasion improved the radiance of the complexion and smoothed the surface of the skin. It also prepared the skin for application of the skin depigmenting composition.

Skin Depigmenting Composition

A skin depigmenting composition in accordance with the present invention was prepared having the following ingredients:

| Ingredients (CTFA Designation) | % w/w |
|---|---|
| Hydroquinone | 2.000 |
| Sodium Hydroxide | 1.500 |
| Ascorbic Acid | 0.050 |
| Propylene Glycol | 6.000 |
| Tetrasodium EDTA | 0.078 |
| Alcohol Denat. | 18.620 |
| Salicylic Acid | 0.200 |
| Sodium Metabisulfite | 0.050 |
| Glycolic Acid | 10.000 |
| Hydroxyethylcellulose | 0.200 |
| Water | q.s 100 |

This composition preferably has a pH of between 2 and 5, advantageously of between 3 and 4.

Skin Peeling Agent in Powder Form

A skin peeling agent, in powder form, in accordance with the present invention was prepared having the following ingredients:

| Ingredients (CTFA Designation) | % w/w |
|---|---|
| Ascorbic Acid | 95.67 |
| Sodium Ascorbate | 0.50 |
| Magnesium Ascorbate/PCA | 0.50 |
| Calcium Ascorbate | 3.70 |
| Sodium Citrate | 0.03 |
| Citric Acid | 0.20 |
| Total | 100.00 |

A pigmentation treating composition was formed, in situ, by mixing 2 scoops (100 mg) of the skin peeling agent, in powder form with 3 droppers full (1.50 ml) of the skin depigmenting composition, in a mixing cup. The pigmentation treating composition was then applied onto the face with a brush.

The pigmentation treating composition makes it possible to close the pores, accelerate skin replacement and lighten the skin color.

Skin Soothing Composition

A skin soothing composition in accordance with the present invention was prepared having the following ingredients:

| Ingredients (CTFA Designation) | % w/w |
|---|---|
| PEG/PPG-18/18 Dimethicone | 1.00 |
| C30-45 Alkyl Dimethicone | 3.00 |
| Preservatives | 1.00 |
| Cyclopentasiloxane | 24.50 |
| Glycerin | 7.00 |
| Propylene Glycol | 3.00 |
| Dimethicone | 1.50 |
| Cetyl Dimethicone | 3.00 |
| Magnesium Sulfate | 1.00 |
| Polysorbate 20 | 0.50 |
| Water | q.s. 100.00 |

The pH of the internal aqueous phase was 5.50.

The skin soothing composition was applied over the pigmentation treating composition and left on the face overnight.

Maintenance Composition

In order to minimize the irritation potential of the above-described compositions, a maintenance composition was used, on alternate days, in place of the above-described compositions.

A maintenance composition in accordance with the present invention was prepared having the following ingredients:

| Ingredients (CTFA Designation) | % w/w |
|---|---|
| Potato Starch Modified | 2.000 |
| Glyceryl Stearate SE | 1.700 |
| Preservatives | 1.013 |
| Hydroquinone | 2.000 |
| Organic Sunscreens | 21.000 |
| Disodium EDTA | 0.050 |
| Potassium Cetyl Phosphate | 2.200 |
| Ethylhexyl Palmitate | 3.000 |
| Cetyl Alcohol | 1.000 |
| Stearic Acid | 1.000 |
| Propylene Glycol | 5.000 |
| Dicaprylyl Ether | 0.500 |
| Cetyl Dimethicone | 1.000 |
| C12-15 Alkyl Benzoate | 4.000 |
| Sodium Metabisulfite | 0.050 |
| Tocopheryl Acetate | 0.200 |
| Capryloyl Salicylic Acid | 0.400 |
| Aluminum Starch Octenylsuccinate | 2.000 |
| Preservatives | 0.237 |
| Water | q.s. 100 |

The pH of the composition was about 5.50 and it had an SPF value of 15.

After a period of use spanning 28 days, a noticeable whitening of the skin was observed.

What is claimed is:

1. A cosmetic kit for treating human skin in need of depigmentation comprising a multi-unit receptacle containing:
(a) at least one unit containing a microdermabrasion composition having at least one type of skin abrading particle;
(b) at least one unit containing a skin depigmenting composition having at least one whitening agent and, optionally, at least one skin peeling agent;
(c) optionally, at least one unit containing at least one skin peeling agent;
(d) at least one unit containing a skin soothing composition having at least one skin soothing agent;
(e) optionally, at least one unit containing a maintenance composition having at least one active chosen from a skin whitening agent and a skin peeling agent; and
(f) optionally, at least one unit containing a photoprotective composition.

2. The kit of claim 1 wherein the microdermabrasion composition contains at least one type of skin abrading particle chosen from metal oxides, ground fruit kernels, diatomaceous earth and pumice.

3. The kit of claim 1 wherein the microdermabrasion composition contains aluminum oxide.

4. The kit of claim 1 wherein the at least one skin whitening agent is hydroquinone.

5. The kit of claim 1 wherein the skin depigmenting composition contains hydroquinone and glycolic acid.

6. The kit of claim 1 wherein the photoprotective composition has an SPF value of at least about 15.

7. The kit of claim 1 further comprising at least one water chosen from thermal water and mineral water, and wherein the at least one water is present in the skin soothing composition and/or the maintenance composition.

8. A method of treating a targeted area of skin in need of depigmentation comprising:
(a) providing a unit containing a microdermabrasion composition having at least one type of skin abrading particle;
(b) providing a unit containing a skin depigmenting composition having at least one skin whitening agent and, optionally, at least one skin peeling agent;
(c) optionally, providing a unit containing at least one skin peeling agent;
(d) providing a unit containing a skin soothing composition having at least one skin soothing ingredient;
(e) optionally, providing a unit containing a photoprotective composition;
(f) contacting the skin with the microdermabrasion composition to form abraded skin;
(g) removing the microdermabrasion composition from the abraded skin;
(h) contacting the skin with the skin depigmenting composition;
(i) applying a layer of skin soothing composition over the skin depigmenting composition;
(j) allowing the skin depigmenting composition and the skin soothing composition to remain on the skin for a predetermined period of time;
(k) removing the skin depigmenting composition and the skin soothing composition from the skin; and
(l) applying a photoprotective composition onto the skin.

9. The method of claim 8 wherein the method is performed daily.

10. The method of claim 8 wherein the method is performed on alternate days.

11. The method of claim 8 wherein the microdermabrasion composition contains at least one type of skin abrading particle chosen from metal oxides, ground fruit kernels, diatomaceous earth and pumice.

12. The method of claim 8 wherein the microdermabrasion composition contains aluminum oxide.

13. The method of claim 8 wherein the at least one skin whitening agent is hydroquinone.

14. The method of claim 8 wherein the at least one skin whitening agent is hydroquinone and glycolic acid.

15. The method of claim 8 further comprising providing a maintenance composition containing at least one active chosen from a skin whitening agent and a skin peeling agent.

16. The method of claim 15 wherein the maintenance composition is applied on the alternate days of claim 10.

17. A cosmetic kit for treating human skin in need of depigmentation comprising a multi-unit receptacle containing:
(a) at least one unit containing a microdermabrasion composition having at least one type of skin abrading particle;
(b) at least one unit containing a depigmenting composition having at least one skin whitening agent, at least one skin peeling agent and at least one skin soothing agent;
(c) optionally, at least one unit containing a maintenance composition having at least one skin whitening agent and, optionally, at least one skin peeling agent; and
(d) optionally, at least one unit containing a photoprotective composition.

18. A method of treating a targeted area of skin in need of depigmentation comprising:
(a) providing at least one unit containing a microdermabrasion composition having at least one type of skin abrading particle;
(b) providing at least one unit containing a depigmenting composition having at least one skin whitening agent, at least one skin peeling agent and at least one skin soothing agent;
(c) optionally, providing at least one unit containing a maintenance composition having at least one skin whitening agent and, optionally, at least one skin peeling agent; and
(d) optionally, at least one unit containing a photoprotective composition;
(e) contacting the skin with the microdermabrasion composition to form abraded skin;
(f) removing the microdermabrasion composition from the abraded skin;
(g) contacting the skin with the skin depigmenting composition;
(h) applying a layer of skin soothing composition over the skin depigmenting composition;
(i) allowing the skin depigmenting composition to remain on the skin for a predetermined period of time;
(j) removing the skin depigmenting composition from the skin; and
(k) applying a photoprotective composition onto the skin.

19. The method of claim 18 further comprising providing a maintenance composition containing at least one active chosen from a skin whitening agent and a skin peeling agent.

20. The method of claim 19 wherein the method is performed daily.

21. The method of claim 19 wherein the method is performed on alternate days.

22. A cosmetic kit for treating human skin in need of depigmentation comprising a multi-unit receptacle containing:
(a) a unit containing a microdermabrasion composition with 20% by weight, based on the weight of the microdermabrasion composition, of aluminum oxide particles;
(b) a unit containing a skin depigmenting composition with 2% by weight of hydroquinone and 10% by weight of glycolic acid, all weights based on the weight of the skin depigmenting composition;
(c) a dropper for dispensing the skin depigmenting composition;
(d) a unit containing a peeling agent, in powder form, with at least 90% by weight, based on the weight of the peeling agent, in powder form, of ascorbic acid;
(e) a scoop for dispensing the peeling agent, in powder form;
(f) a mixing cup for blending the skin depigmenting composition and the peeling agent, in powder form, to form a pigmentation treating composition;
(g) a brush for applying the blended composition onto a targeted area of skin;
(h) a unit containing a skin soothing composition having at least one skin soothing ingredient;
(i) a unit containing a maintenance composition having at least one active chosen from a skin whitening agent and a peeling agent in a concentration less than that of the skin depigmenting composition, optionally having at least one sunscreen active; and
(j) optionally, a unit containing a photoprotective composition having an SPF value of at least about 15.

23. The kit of claim 22 further comprising at least one water chosen from thermal water and mineral water, and wherein the at least one water is present in the skin soothing composition and/or the maintenance composition.

24. A method of treating a targeted area of skin in need of depigmentation comprising:
(a) providing a unit containing a microdermabrasion composition with 20% by weight, based on the weight of the microdermabrasion composition, of aluminum oxide particles;
(b) providing a unit containing a skin depigmenting composition with 2% by weight of hydroquinone and 10% by weight of glycolic acid, all weights based on the weight of the skin depigmenting composition;
(c) providing a unit containing a skin peeling agent, in powder form, with at least 90% by weight, based on the weight of the skin peeling agent, of ascorbic acid;
(d) providing a unit containing a skin soothing composition having at least one skin soothing agent;
(e) providing a unit containing a photoprotective composition;
(f) contacting the skin with the microdermabrasion composition to form abraded skin;
(g) removing the microdermabrasion composition from the abraded skin;
(h) mixing the skin depigmenting composition and the skin peeling agent, in powder form, to form a pigmentation treating composition;
(i) contacting the skin with the pigmentation treating composition;
(j) applying a layer of skin soothing composition over the pigmentation treating composition;
(k) allowing the pigmentation treating composition and the skin soothing composition to remain on the skin for a predetermined period of time;
(l) removing the pigmentation treating composition and the skin soothing composition from the skin; and
(m) applying a photoprotective composition onto the skin.

25. The method of claim 24 wherein the method is performed daily.

26. The method of claim 24 wherein the method is performed on alternate days.

27. The method of claim 26 further comprising providing a maintenance composition containing at least one active chosen from a skin whitening agent wherein the active is present in the maintenance composition in an amount less than that in the skin depigmenting composition.

28. The method of claim 27 wherein the method is preformed on alternate days of claim 26.
